(19) United States
(12) Patent Application Publication
(10) Pub. No.: US 2002/0146666 A1
(43) Pub. Date: Oct. 10, 2002

(54) Kits for whitening teeth

(76) Inventors: Paul Albert Sagel, Mason, OH (US); Melissa Ellen Jeffers, Wyoming, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 09/825,733

(22) Filed: Apr. 4, 2001

(51) Int. Cl. A61C 5/00

(52) U.S. Cl. 433/215

(57) ABSTRACT

A kit for whitening teeth is provided. The kit includes a package containing a tooth whitening composition with a peroxide active and instructions in association with the package for minimizing soft tissue irritation comprising an instruction not to brush the teeth and/or soft tissue prior to applying the tooth whitening composition.

### Technical Field of the Invention

The present invention relates to kits for whitening teeth, and, more particularly, to kits having instructions for minimizing soft tissue irritation caused by a peroxide active in a tooth whitening composition.

### BACKGROUND OF THE INVENTION

Tooth whitening has become very popular over the past few years. More and more consumers are choosing to whiten their teeth. Options for tooth whitening include toothpastes, mouthrinses, chewing gums, in-office bleaching, and most commonly tooth whitening solutions used with a tray obtained either over-the-counter or from a dentist. The tooth whitening solutions contain an active ingredient which bleach the teeth, such as hydrogen peroxide.

Hydrogen peroxide is typically not damaging to the tissue in the range of concentrations used for whitening (0-15% hydrogen peroxide), because saliva contains enzymes (peroxidase, amalase and catalase) which rapidly turn peroxide into oxygen and water before the peroxide can significantly react with any other substances. While these enzymes are also present on the surface of soft tissue, brushing can wash away the enzymes leaving the soft tissue susceptible to irritation from the peroxide and a resulting burning sensation to the user. In addition, brushing can also produce abrasions in the soft tissue. When hydrogen peroxide is applied to these abrasions, a burning sensation develops similar to pouring peroxide into an open wound. If repeated brushings occur during a day, the abrasions can build over time as they do not have sufficient time to heal.

Recent developments in the tooth whitening field have seen the introduction of tooth whitening systems which result in the application of some of the tooth whitening composition to the soft tissue as described in U.S. Pat. No. 6,136,297. As such, there is a continuing desire to provide tooth whitening kits having instructions which communicate the desirability of not brushing the teeth prior to application of a tooth whitening composition, especially where a peroxide active is applied to the soft tissue as part of the whitening process. In addition, where brushing is desirable prior to application of a tooth whitening composition, there is a need to provide instructions communicating the parameters associated with this brushing.

If brushing does occur before application of a tooth whitening composition, the pH of the toothpaste can be important. For example, toothpaste pH can impact subsequent formation of radicals when a tooth whitening composition is applied to the teeth. A high pH can facilitate formation of radicals resulting in increased gum irritation during the whitening process. As such, there is also a desire to provide tooth whitening kits which include an appropriate toothpaste if it is desired to brush the teeth prior to application of a tooth whitening composition.

### SUMMARY OF THE INVENTION

A kit for whitening teeth is provided. The kit includes a package containing a tooth whitening composition with a peroxide active and instructions in association with the package for minimizing soft tissue irritation comprising an instruction not to brush the teeth and/or soft tissue prior to applying the tooth whitening composition. The kit can further include an instruction which communicates that the teeth and/or soft tissue should not be brushed between 0 and about 60 minutes prior to applying the tooth whitening composition to the teeth. The kit may optionally contain toothpaste and a toothbrush for brushing the teeth after application of the tooth whitening composition.

### BRIEF DESCRIPTION OF THE DRAWINGS

While the specification concludes with claims particularly pointing out and distinctly claiming the invention, it is believed that the present invention will be better understood from the following description taken in conjunction with the accompanying drawings in which:

FIG. 1 is a perspective view of a kit made in accordance with the present invention;

FIG. 2 is a perspective view of a tooth whitening system suitable for use with the kit of FIG. 1;

FIG. 3 is cross-sectional side elevational view of the tooth whitening system of FIG. 2, taken along line 2-2 thereof;

FIG. 4 is a top planar view of another preferred strip suitable for use with the tooth whitening system of FIG. 2;

FIG. 5 is a top planar view of yet another preferred strip suitable for use with the tooth whitening system of FIG. 2;

FIG. 6 is a top planar view of still yet another preferred strip suitable for use with the tooth whitening system of FIG. 2;

FIG. 7 is a schematic top plan view of the tooth whitening system of FIG. 2 applied to the maxillary teeth; and

FIG. 8 is cross-sectional side elevational view of the maxillary teeth of FIG. 7, taken along line 8-8 thereof.

### DETAILED DESCRIPTION OF THE PREFERRED EMBODIMENTS

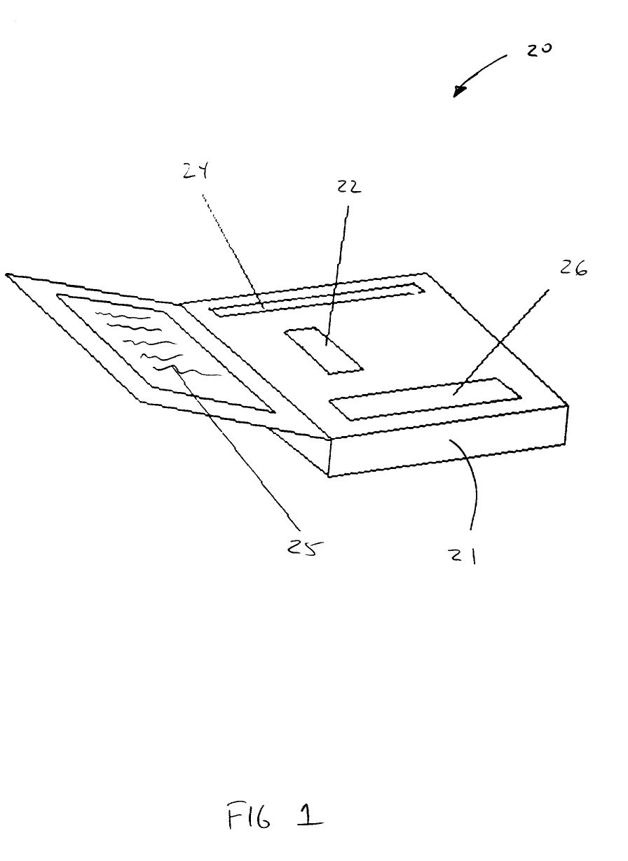

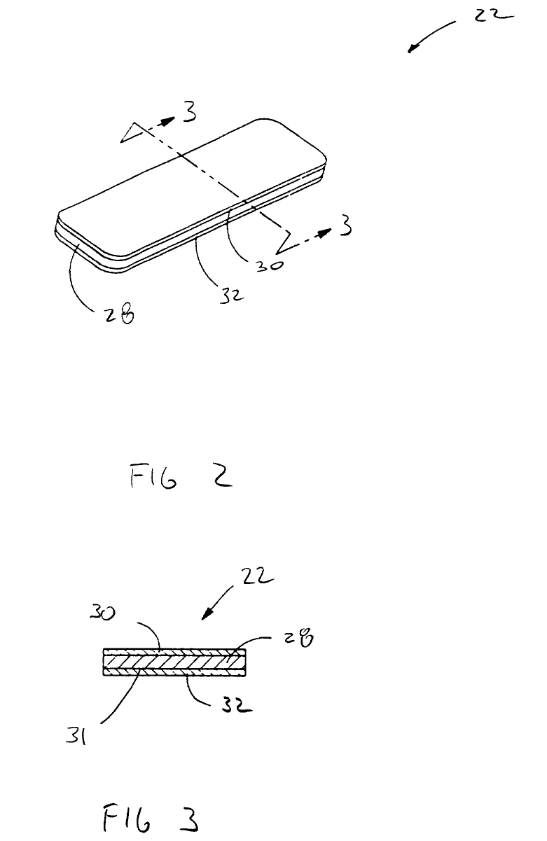

Reference will now be made in detail to the present preferred embodiments of the invention, examples of which are illustrated in the accompanying drawings wherein like numerals indicate the same elements throughout the views. Referring to FIGS. 1, 2, and 3 and in accordance with one aspect of the present invention, an embodiment of the a kit 20 made in accordance with the present invention will now be described, wherein the kit 20 comprises a package 21 for storing a tooth whitening system 22, instructions 25 in association with the kit 20, and optionally a toothbrush 24 and toothpaste 26. The tooth whitening system 22 can include a tooth whitening composition 28 containing a peroxide active and an applicator which applies and/or maintains application of the tooth whitening composition to the teeth and/or soft tissue. An exemplary tooth whitening system 22 is illustrated in FIGS. 2 and 3 and comprises a strip of material 30, a thin layer of a tooth whitening composition 28, and optionally a release liner 32 disposed opposite the strip of material 30 which covers the surface 31 of the tooth whitening composition which is applied to the teeth using the strip of material. The kit 20 can comprise a plurality of tooth whitening systems 22 for multiple applications of the tooth whitening composition.

The tooth whitening composition 22 may be pre-applied to the applicator, or applied to the applicator by the user, or applied to the teeth and soft tissue by the user and then covered by applicator. Exemplary tooth whitening compositions containing a peroxide active are disclosed in U.S. Pat. Nos. 6,136,297; 5,376,006; 5,985,249; 5,922,307; RE 34,196; and WO 99/62472, the substances of which are incorporated by reference. Peroxide actives suitable for use with the present invention include hydrogen peroxide, calcium peroxide, carbamide peroxide, and mixtures thereof. Most preferred is hydrogen peroxide. Other peroxide actives include compositions which produce hydrogen peroxide when mixed with water, such as the percarbonates, specifically sodium percarbonate. While the peroxide active can be present in any concentration, it is preferred that the peroxide active is present in an concentration between about 0.01% and about 40%, by weight of the tooth whitening composition in tooth whitening applications. The peroxide active should provide a concentration of hydrogen peroxide equivalent between about 0.1% and about 20%, preferably between about 0.5% and about 15%, more preferably between about 1% and about 10%, and most preferably between about 2% and about 10% by weight of the tooth whitening composition. It is understood that these concentrations are expressed for hydrogen peroxide and appropriate conversions must be made for other peroxide liberating molecules such as carbamide peroxide, calcium peroxide, etc.

Additional constituents of the tooth whitening composition can include, but are not limited to, water, gelling agents, humectants, pH adjusting agents, stabilizing agents, desensitizing agents, and accelerating agents or bleach activators. In addition to the above materials, a number of other materials can also be added to the tooth whitening composition. Additional materials include, but are not limited to, flavoring agents, sweetening agents such as saccharin, xylitol, opacifiers, coloring agents, and chelants such as ethylenediaminetetraacetic acid.

Suitable gelling agents do not react with or inactivate the constituents of the tooth whitening composition. Some gelling agents useful in the present invention include “Pemulen” made by B. F. Goodrich Company, carboxypolymethylene, carboxymethyl cellulose, carboxypropyl cellulose, hydroxyethyl cellulose, poloxamer, Laponite, carrageenan, Veegum, carboxyvinyl polymers, and natural gums such as gum karaya, xanthan gum, Guar gum, gum arabic, gum tragacanth, and mixtures thereof. The preferable gelling agent for use in the present invention is carboxypolymethylene, obtained from B. F. Goodrich Company under the tradename CARBOPOL®. Particularly preferred CARBOPOLs include CARBOPOL® 934, 940, 941, 956, 971, 974, 980, and mixtures thereof. Particularly preferred is CARBOPOL® 956. Carboxypolymethylene is a slightly acidic vinyl polymer with active carboxyl groups. The amount of the gelling agent can be between about 0.1% and about 15%, preferably between about 1% and about 10%, more preferably between about 2% and about 8%, and most preferably between about 3% and about 6%, by weight of the tooth whitening composition. An effective concentration of a poloxamer gelling agent is between about 10% and about 40%, preferably between about 20% and about 35%, and more preferably between about 25% and about 30%, by weight of the tooth whitening composition.

Other suitable gelling agents include both polymers with limited water solubility as well as polymers lacking water solubility. Suitable limited water solubility adhesives include: hydroxy ethyl or propyl cellulose. Adhesives lacking water solubility include: ethyl cellulose and polyox resins. Another possible adhesive suitable for use in the instant composition is polyvinylpyrrolidone with a molecular weight between about 50,000 and about 300,000. Still another possible adhesive suitable for use in the instant composition is a combination of Gantrez and the semisynthetic, water-soluble polymer carboxymethyl cellulose.

A pH adjusting agent may also be added to make the tooth whitening composition safe for oral tissues. These pH adjusting agents, or buffers, can be any material which is suitable to adjust the pH of the tooth whitening composition. Suitable materials include sodium bicarbonate, sodium phosphate, sodium hydroxide, ammonium hydroxide, potassium hydroxide, sodium stannate, triethanolamine, citric acid, hydrochloric acid, sodium citrate, and combinations thereof. The pH adjusting agents are added in sufficient concentrations so as to adjust the pH of the tooth whitening composition to between about 3 and about 10, preferably between about 4 and about 8.5, and more preferably between about 4.5 and about 8. The pH adjusting agents are generally present in an concentration between about 0.01% and about 15% and preferably between about 0.05% and about 5%, by weight of the tooth whitening composition.

Suitable stabilizing agents include benzoic acid, salicylic acid, butylated hydroxytoluene, tin salts, phosphates, and others. Suitable bleach activators include trichloroisocyanuric acid and the phosphates, such as tetrasodium pyrophosphate.

Desensitizing agents may also be used in the tooth whitening composition. These agents may be preferred for consumers who have sensitive teeth. Desensitizing agents include potassium nitrate, citric acid, citric acid salts, strontium chloride, and combinations thereof. Potassium nitrate is a preferred desensitizing agent. Other agents which provide the benefit of reduced tooth sensitivity are also included in the present invention. Typically, the concentration of a desensitizing agent is between about 0.01% and about 10%, preferably between about 0.1% and about 8%, and more preferably between about 1% and about 7% by weight of the tooth whitening composition.

The tooth whitening composition can be applied to the teeth using a variety of applicators. For example, the tooth whitening composition can be applied using a tray, stint, or similar device, such as described in U.S. Pat. Nos. 5,376,006 and RE 34,196. In yet other embodiments, the tooth whitening composition can be applied using a brush, pen applicator, doe's foot applicator or by finger, cotton swab, or dental stick or the like.

A particularly preferred applicator is provided in the form of a strip of material, such as described in U.S. Pat. Nos. 5,891,453; 6,136,297; 5,879,691; and 5,989,569 the substances of which are incorporated herein by reference. The strip of material is used to apply the tooth whitening composition to the teeth and to act as a protective barrier for the tooth whitening composition. It prevents substantial leaching and/or erosion of the tooth whitening composition by, for example, the wearer's lips, tongue, and saliva. This allows the peroxide active of the tooth whitening composition to act upon the teeth for an extended period of time.

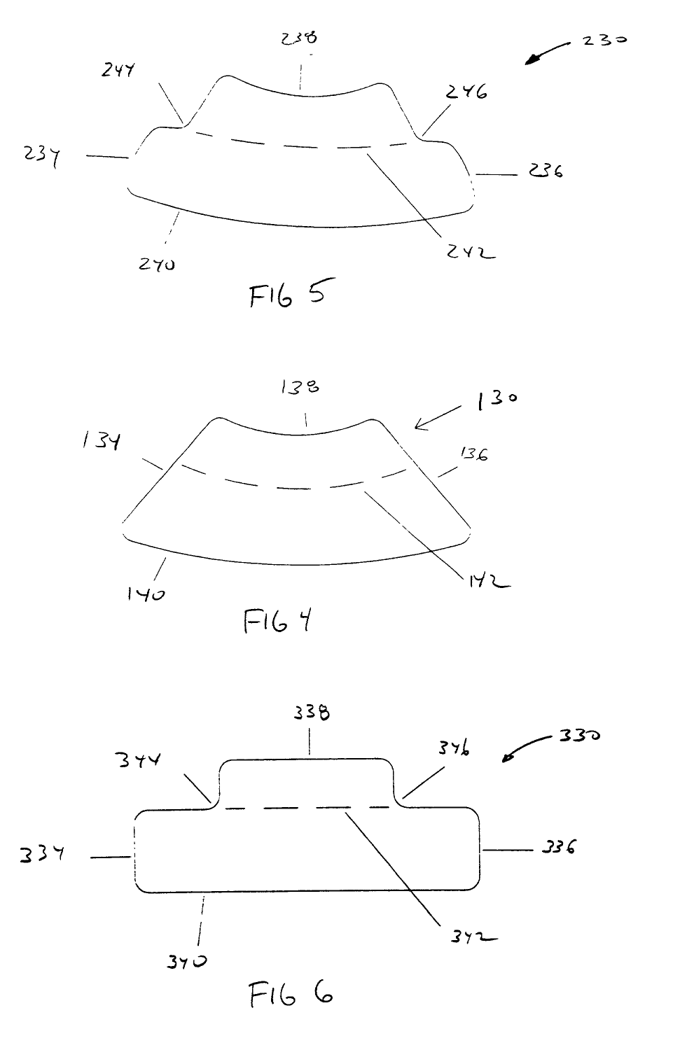

Referring to FIGS. 2 to 6, exemplary strips suitable for use with the tooth whitening system 22 are illustrated. While the strip of material can be provided in the substantially rectangular shape illustrated in FIG. 2, it is contemplated that the strip of material 30 can also be provided in a substantially trapezoidal shape as show in FIG. 4. The strip of material 130 has a first side 134 and second side 136, a third side 138, and a fourth side 140. First side 134 and second side 136 are straight sides which angle in from the fourth side 140 to the third side 138. Third side 138 is concave and shorter then the fourth side 140. The fourth side 140 is convex. The fourth side 140 will be placed close to the bottom edge of the front side of a user's bottom set of front teeth. Alternatively, if the strip is worn on the user's top set of teeth, the fourth side 140 may be placed along the top part of the front side of a user's top set of front teeth. A fold line 142 of the strip of material 130 extends from first side 134 to second side 136. The fold line 142 may be located closer to the third side 138 or the fourth side 140. The third side 138 will be along the back side of a user's teeth after the strip of material 130 is folded along the fold line 142. Referring to FIG. 5, an alternate strip of material 230 is illustrated which is substantially trapezoidal in shape with stair-stepped sides. Fold line 242 extends from the corners 244 and 246 of the stair step in the first side 234 and the second side 236, respectively. The strip of material 230 can alternatively be described as two substantially trapezoidal shapes placed on top of one another. Referring to FIG. 6, another alternate strip of material 330 is illustrated having stair-stepped sides. The strip of material 330 can also be described as two rectangles placed on top of one another. As will be appreciated, other shapes for the strip of material can be provided.

The strip of material should be conformable to the contoured surfaces of the teeth, the interstitial tooth spaces, and the soft tissue. A relatively low flexural stiffness enables the strip of material to drape over the contoured surfaces of teeth and soft tissue with very little force being exerted; that is, conformity to the curvature of the wearer's mouth and gaps between adjacent teeth is maintained because there is little residual force within strip of material to cause it to return to its substantially flat shape. The flexibility of the strip enables the strip of material to contact adjoining soft tissue over an extended period of time without physical irritation.

Flexural stiffness is a material property that is a function of a combination of strip thickness, width, and material modulus of elasticity. This test is a method for measuring the rigidity of polyolefin film and sheeting. It determines the resistance to flexure of a sample by using a strain gauge affixed to the end of a horizontal beam. The opposite end of the beam presses across a strip of the sample to force a portion of the strip into a vertical groove in a horizontal platform upon which the sample rests. A microammeter, wired to the strain gauge is calibrated in grams of deflection force. The rigidity of the sample is read directly from the microammeter and expressed as grams per centimeter of sample strip width. In the present invention, the strip of material has a flexural stiffness of less than about 5 grams/cm as measured on a Handle-O-Meter, model #211-300, available from Thwing-Albert Instrument Co. of Philadelphia, Pa., as per test method ASTM D2923-95. Preferably, the strip of material has a flexural stiffness less than about 4 grams/cm, more preferably less than about 3 grams/cm, and most preferably from about 0.1 grams/cm to about 1 grams/cm. Preferably, the flexural stiffness of the strip of material is substantially constant and does not significantly change during normal use. For example, the strip of material does not need to be hydrated for the strip to achieve the low flexural stiffness in the above-specified ranges. The strip of material may be formed by any of several film making processes known in the art. Preferably, a strip of material made of polyethylene is made by a blown process or a cast process. Processes, such as extrusion and other processes, may also be suitable.

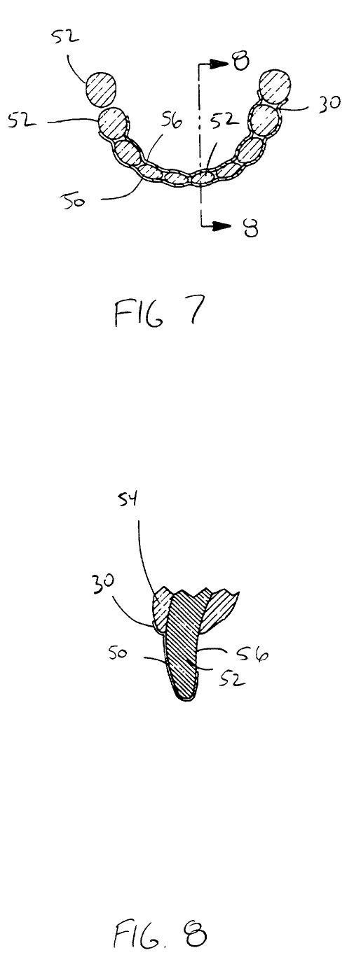

Referring to FIGS. 7 and 8, the strip of material is preferably sized and configured to cover a portion of the teeth to be whitened and portions of the soft tissue adjacent these teeth such that the tooth whitening composition is applied to both the teeth and soft tissue. The strip of material is held in place on a plurality of adjacent teeth by adhesive attachment to the teeth and/or the soft tissue, wherein the adhesiveness can be provided by one or more of the tooth whitening compositions described herein. Soft tissue is herein defined as soft tissues surrounding the tooth structure, including papilla, marginal gingival, gingival sulculus, inter dental gingival, gingival gum structure on lingual and buccal surfaces up to and including muco-ginival junction and the pallet. The viscosity and general adhesiveness of the tooth whitening composition cause the strip of material to be adhesively attached to a plurality of adjacent teeth and/or soft tissue without substantial slippage under the potential friction from the lips, tongue, and other soft tissue rubbing against the strip of material during mouth movements associated with talking, drinking, etc. However, this adhesion to the teeth and/or soft tissue is low enough to allow the strip of material to be easily removed by the wearer by peeling off the strip of material using one's finger or fingernail.

In an alternate embodiment, the strip is sized and configured to cover all of the front tooth surface 50 of one or more teeth 52, a portion of the soft tissue 54 adjacent the front tooth surface 50, and a portion of rear tooth surface 56, as shown in FIG. 8. In general, the length of the strip of material is from about 2 cm to about 12 cm, preferably from about 3 cm to about 9 cm, and more preferably from about 4 cm to about 8 cm. If the strip is stair stepped on the sides or trapezoidal shaped, the longer side of the strip is generally from about 2 cm to about 12 cm, preferably from about 3.1 to about 10 cm, more preferably from about 3.5 cm to about 8 cm, and most preferably from about 4 cm to about 6 cm. The shorter side is from about 0.1 cm to about 12 cm, preferably from about 0.5 cm to about 8 cm, more preferably from about 1 cm to about 5 cm, and most preferably from about 1.5 cm to about 3 cm. The width of the strip will also depend upon many factors, including whether or not the strip of material wraps completely around the teeth and covers part or all of the front and back surfaces of the tooth. In a general application, the width of the strip is between about 0.5 cm and about 4 cm and preferably between about 1 cm and about 2 cm.

The optional release liner 32 may be formed from any material which exhibits less affinity for the tooth whitening composition than the tooth whitening composition exhibits for itself and for the strip of material. The release liner preferably comprises a rigid sheet of material such as polyethylene, paper, polyester, or other material which is then coated with a non-stick type material. The release liner material may be coated with wax, silicone, polyester such as Teflon®, fluoropolymers, or other non-stick type materials. A preferred release liner is SCOTCHPAK®, produced by Minnesota Mining and Manufacturing Company, Minneapolis, Minn. The release liner may be cut to substantially the same size and shape as the strip of material or the release liner may be cut larger than the strip of material to provide a readily accessible means for separating the release liner from the strip of material.

In one method of use, the release liner 32 is removed from the tooth whitening system 22, thereby exposing the surface 31 of the thin layer of the tooth whitening composition 28. The strip of material 30 is then used to apply the exposed tooth whitening composition 28 to the teeth. The fold line is placed over the tips of a user's teeth enabling the strip of material 30 to fold down onto both the front tooth surface 50 of the user's teeth 52 and a portion of the soft tissue adjacent the front tooth surface 50 as well as the back surface 56 of the user's teeth. For the stair-stepped strips, the strip of material will be placed so that the user's two canines are just outside of corners of the strip. The fourth side will be located close to the bottom edge of the front side of a user's bottom set of front teeth, as seen in FIG. 8. Alternatively if the strip of material is worn on the user's top set of teeth, the fourth side may be placed along the top part of the front side of a user's top set of front teeth. The third side will be along the back side of a user's teeth.

The kit 20 may optionally also include an oral composition, such as a toothpaste. While a variety of oral compositions are suitable for use with the kits of the present invention, some examples are described in U.S. Pat. Nos. 4,254,101; 4,314,990, 4,842,165; 4,515,772; 5,004,597; 5,885,556; 5,820,852; 5,849,269; 5,451,404; 5,820,854; and 5,939,052, the substances of which are incorporated herein by reference. The kit 20 may also optionally include a toothbrush for use with the oral composition. While a variety of toothbrush are suitable, some examples are described in U.S. Pat. Nos. 6,178,583; 6,115,870; 5,926,897; 5,839,148; 5,511,275; and 5,165,761, the substances of which are incorporated herein by reference. Preferably, the pH of the oral composition is between about 2 and about 9 and more preferably between about 3 and about 8. A pH within these ranges can minimize the formation of OOH⁻ radicals during the whitening process, thereby reducing gum irritation.

In accordance with one aspect of the present invention, a set of instructions 25 in the form of text, graphics, or a combination thereof are provided in association with the kit 20. As used herein, the phrase “in association with” means that the set of instructions are either directly printed on a package of the kit, a component of the kit (e.g., the tooth whitening system container, the toothpaste container, etc.) or presented in a separate manner including, but not limited to, a brochure, print advertisement, electronic advertisement, and/or verbal communication, so as to communicate the set of instructions to a consumer. The instructions communicate the instruction not to brush the teeth and/or soft tissue prior to applying the tooth whitening composition. In an alternate embodiment, the instructions further communicate that the teeth and/or soft tissue should not be brushed immediately before application of the tooth whitening composition. In yet another alternate embodiment, the instructions further communicate that the teeth and/or soft tissue should not be brushed between about 0 and about 60 minutes prior to applying the tooth whitening composition to the teeth. More preferably, the instructions communicate that the teeth and/or soft tissue should not be brushed between about 0 and about 30 minutes prior to applying the tooth whitening composition to the teeth. Most preferably, the instructions communicate that the teeth should not be brushed between about 0 and about 15 minutes before applying the tooth whitening composition to the teeth. In order to minimize the accumulation of abrasions, the instructions can communicate that the teeth should be brushed no more than one time per day when using the tooth whitening composition. In an alternate embodiment, the instructions can communicate that the teeth should be brushed less than or equal to two times per day when using the tooth whitening composition.

While particular embodiments of the present invention have been illustrated and described, it will be obvious to those skilled in the art that various changes and modifications may be made without departing from the spirit and scope of the invention, and it is intended to cover in the appended claims all such modifications that are within the scope of the invention.

What is claimed is:

1. A kit for whitening teeth, comprising:
a package containing a tooth whitening composition comprising a peroxide active; and
an instruction in association with said package for minimizing soft tissue irritation comprising an instruction not to brush the teeth prior to applying said tooth whitening composition.

2. The kit of claim 1, wherein said instruction further communicates that the teeth should not be brushed between about 0 and about 60 minutes prior to applying said tooth whitening composition to the teeth.

3. The kit of claim 1, wherein said instruction further communicates that the teeth should not be brushed between about 0 and about 30 minutes prior to applying said tooth whitening composition to the teeth.

4. The kit of claim 1, wherein said instruction further communicates that the teeth should not be brushed between about 0 and about 15 minutes prior to applying said tooth whitening composition to the teeth.

5. The kit of claim 1, further comprising a toothpaste.

6. The kit of claim 5, further comprising a toothbrush.

7. The kit of claim 6, wherein said instruction further communicates to use said toothpaste and said toothbrush after application of said tooth whitening composition.

8. The kit of claim 5, wherein said toothpaste has a pH between about 2 and about 9.

9. The kit of claim 5, wherein said toothpaste has a pH between about 3 and about 8.

10. The kit of claim 1, further comprising an applicator for said tooth whitening composition.

11. The kit of claim 10, wherein said applicator is a strip of material.

12. The kit of claim 10, wherein said applicator is a tray.

13. The kit of claim 10, wherein said applicator is sized to overlap the soft tissue.

14. The kit of claim 10, further comprising a plurality of said applicators.

15. The kit of claim 1, wherein said instruction further communicates that the teeth should be brushed less than or equal to two times per day when using said tooth whitening composition.

16. The kit of claim 1, wherein said instructions are printed on said package.

17. A kit for whitening teeth, comprising:
a package containing a tooth whitening composition comprising a peroxide active; and
an instruction in association with said package for minimizing soft tissue irritation comprising an instruction not to brush the teeth immediately before applying said tooth whitening composition to the teeth.

18. The kit of claim 17, further comprising a toothpaste having a pH between about 2 and about 9.

19. The kit of claim 17, wherein said toothpaste has a pH between about 3 and about 8.

20. The kit of claim 17, further comprising an applicator for said tooth whitening composition.
